(19) United States
(12) Patent Application Publication
(10) Pub. No.: US 2010/0129327 A1
(43) Pub. Date: May 27, 2010

(54) METHOD OF TREATING HEARING LOSS

(76) Inventors: Sharon Oleskevich, Coogee (AU); Kharen Doyle, Drummoyne (AU); Sarah McKay, Mosman (AU)

Correspondence Address: WILMERHALE/BOSTON, 60 STATE STREET, BOSTON, MA, 02109, US

(21) Appl. No.: 12/444,759

(22) PCT Filed: Oct. 10, 2007
(86) PCT No.: PCT/AU2007/001534

§ 371 Date: Feb. 8, 2010

Related U.S. Application Data
(60) Provisional application No. 60/851,214, filed on Oct. 11, 2006.

(51) Int. Cl. A61K 35/12; C12N 5/00; A61P 27/16

(52) U.S. Cl. 424/93.7; 435/377; 435/325

(57) ABSTRACT

The present invention relates to a method of generating cochlear hair cell-like cells in the inner ear of a subject. Preferably, the subject is human and is suffering from sensorineural hearing loss. The invention also relates to a method of generating cochlear hair cell-like cells in vitro and use of the cochlear hair cell-like cells for treating or preventing sensorineural hair loss in a subject.

### FIELD OF THE INVENTION

The present invention relates to methods of generating cochlear hair cell-like cells in vivo and in vitro. The methods of the invention are useful in the treatment of inner hair cell related conditions, for example, sensorineural hearing loss.

### BACKGROUND OF THE INVENTION

Sensorineural deafness induced by ageing, excessive noise and certain antibiotics accounts for the majority of permanent hearing loss in humans. Hearing loss is caused by the dysfunction of the sensory epithelium (the organ of Corti) within the inner ear (cochlea). It is associated with the irreversible loss of sensory hair cells (Raphael, 2002) and spiral ganglion neurons (Raphael, 2002). There is evidence of regeneration of sensory cells in non-mammalian species (Stone and Rubel, 2000; Morest and Cotanche, 2004), however, studies in mammals have failed to find evidence of cochlear regeneration (Forge and Nevill, 1998).

Several areas of research have addressed the treatment of sensorineural hearing loss. Gene therapy has been applied to protect or generate cochlear hair cells. The insertion and overexpression of neurotrophic factors or oxidative stress-reducing enzymes by viral transfection protects cochlear hair cells from ototoxic trauma in guinea pigs (Kawamoto et al., 2003a; Kawamoto et al., 2004). The overexpression of a transcription factor essential for hair cell development in nonsensory cochlear cells, Math1/Atoh1, via viral transfection generates new hair cells and substantially improves hearing thresholds in adult deaf guinea pigs (Kawamoto et al., 2003b; Izumikawa et al., 2005). Other treatments for hearing loss include the exogenous delivery of neurotrophic factors in concert with electrical stimulation to increase the survival of spiral ganglion neurons (Shepherd et al., 2005; Gillespie and Shepherd, 2005). White et al. (2006) have shown that post-mitotic mammalian supporting cells (p27^{Kip1}-positive) have the ability to divide and trans-differentiate into hair cells.

Stem cell therapy has recently been investigated as a potential treatment for hair cell loss. Mouse embryonic stem cells can differentiate into hair cells in the developing inner ear of chick embryos (Li et al., 2003a), while murine neural stem cells (C17.2) in a mouse model have been shown to differentiate into cochlear hair cells (US 2003/0114381).

The potential for stem cell therapy for treatment of sensorineural hearing loss in humans, in particular the use of stem cells originating from human embryos or aborted foetal tissues is complicated by moral and ethical considerations. To avoid such debate, some researchers have investigated the potential of adult stem cells for the treatment of sensorineural loss. Notably, such use of adult stem cells also provides an alternative to xenografts in addition to avoiding tumorogenic complications associated with undifferentiated embryonic stem cells. Adult stem cells, isolated from the mouse vestibular system, can differentiate into hair cells in the developing inner ear of chick embryos (Li et al., 2003b), while adult neural stem cells survive better and differentiate to a greater extent in deafened versus normal guinea pig cochlea (Hu et al., 2005). Some reports show the presence of limited numbers of nestin-positive stem cells in intact mouse organ of Corti (Lopez et al., 2004), and dissociated neonatal rat organ of Corti (Malgrange et al., 2002), suggesting that there may be some intrinsic potential for repair.

While the possibility of deriving stem cells from adult tissue sources holds real promise, there are also some significant limitations. First, adult stem cells are often present in minute quantities, are difficult to isolate and purify, and their number may decrease with age. Further, although stem cells have been isolated from the adult central nervous system, it is not currently possible to remove them without serious consequences to the donor. Accordingly, there is a need for an improved method of stem cell therapy for the treatment of sensorineural hearing loss.

### SUMMARY OF THE INVENTION

The present inventors have now made the surprising finding that adult olfactory precursor cells can differentiate into cochlear hair cell-like cells in vitro. Importantly, human adult olfactory stem cells have recently been shown to be readily obtainable from the nasal cavity without permanent damage to the donor individual (WO 03/064601). The olfactory neuroepithelium therefore offers an abundant and easily accessible source of adult stem cells, a significant advantage for future autotransplantation cell therapy. Adult olfactory precursor cells are therefore potentially useful for the treatment of inner hair cell related conditions, for example, sensorineural hearing loss.

Accordingly, the present invention provides a method of generating cochlear hair cell-like cells in the inner ear of a subject comprising administering adult olfactory precursor cells or progeny thereof to the inner ear of the subject.

In a preferred embodiment of the present invention the subject is human. In a further embodiment, the human is suffering sensorineural hearing loss.

In a further preferred embodiment of the invention, the adult olfactory precursor cells are autologous.

In a further preferred embodiment of the invention, the adult olfactory precursor cells are isolated from the nasal cavity.

In a further preferred embodiment of the invention, the progeny of adult olfactory precursor cells are cells that cluster together to form neurospheres.

In a further preferred embodiment of the invention, the adult olfactory precursor cells or progeny thereof are administered to the cochlea. Preferably the adult olfactory precursor cells or progeny thereof are suspended in a buffer prior to administration.

In one embodiment of the invention, the adult olfactory precursor cells or progeny thereof are administered to the cochlea by contact with the scala tympani via round window penetration.

In another embodiment of the invention, the adult olfactory precursor cells or progeny thereof are administered to the cochlea through the external auditory canal and middle ear.

The present invention also provides a method of generating cochlear hair cell-like cells in vitro comprising culturing adult olfactory precursor cells or progeny thereof with cochlear cells or supernatant derived therefrom, such that the olfactory precursor cells or progeny thereof differentiate into cochlear hair cell-like cells.

In a preferred embodiment of this method, the adult olfactory precursor cells or progeny thereof and the cochlear cells or supernatant derived therefrom are co-cultured for 7 to 14 days.

Preferably the adult olfactory precursor cells or progeny thereof and the cochlear cells are separated during the culturing phase to prevent direct contact between the adult olfactory precursor cells or progeny thereof and the cochlear cells. For example, the adult olfactory precursor cells and cochlear cells may be separated by way of a transwell culture system.

In a preferred embodiment of the invention, the cochlear hair cell-like cells generated by this method of culture are characterised as Myosin VIIa, calretinin, phalloidin and epsin positive and β III tubulin negative.

In a further preferred embodiment of the invention, the cochlear hair cell-like cells generated by this method of culture are also characterised with electro-physiological recordings.

The present invention also provides cochlear hair cell-like cells obtained by the method of culturing of the present invention.

The present invention also provides a method of treating or preventing an inner hair cell related condition comprising administering to the inner ear of a subject adult olfactory precursor cells or progeny thereof.

The present invention also provides a method of treating or preventing an inner hair cell related condition comprising administering to the inner ear of a subject cochlear hair cell-like cells obtained by the method of culturing of the present invention.

In one embodiment of the invention, the inner hair cell related condition is sensorineural hearing loss.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE ACCOMPANYING DRAWINGS

FIG. 1. Olfactory neurospheres show hallmarks of precursor cells. (A): Olfactory neurospheres consist of three-dimensional clusters of densely packed olfactory precursor cells after 24-48 hours in culture. (B): Olfactory neurospheres are self-renewing as shown by incorporation of BrdU and fluorescent staining of individual nuclei within the olfactory neurosphere. Olfactory neurospheres spontaneously differentiated into (C): two morphologically different types of neuron, stellate (arrows) and spherical soma (arrowheads) that were immunopositive for β III tubulin; (D): neuroepithelial cells that were immunopositive for keratin; (E): olfactory cells of non-neuronal lineage (sustentacular, glandular, or basal cells) that were immunopositive for Pax-6; and (F): glial cells that were immunopositive for GFAP. Scale bars=20 μm in (A-D); 10 μm in (E-F). Abbreviations: BrdU, bromodeoxyuridine; GFAP, glial fibrillary-associated protein.

FIG. 2. Characterisation of hair cells in adult mouse organ of Corti. (A): Photomicrograph of cross section of organ of Corti showing positive immunoreactivity for calretinin in the inner hair cell (arrowhead) and auditory nerve fibres (white arrow) but not outer hair cells (arrows). (B): Positive immunoreactivity for prestin in outer hair cells only (arrows). The inner hair cell is negative (arrowhead). (C): Double labelling immunohistochemistry showing distinct labelling of inner hair cells with calretinin (arrowhead), and outer hair cells with prestin (arrows). (D): Myosin VIIa immunofluorescence is seen in the cytoplasm of the inner hair cell (arrowhead). Scale bars=10 μm in (A-D).

FIG. 3. Characterisation of hair cells in primary inner ear cultures using specific hair cell markers. (A): Phase contrast photomicrograph showing different morphologies of cell types within primary adult cochlear cultures. In particular, there are pear-shaped cells (black arrow) and bipolar cells (white arrow) with stereociliary tufts (arrowheads). (B): Fluorescence immunolabelling of Math1-positive nuclei in hair cells after 6 days in culture. (C): Fluorescence immunocytochemistry showing myosin VIIa-positive cytoplasmic staining of a bipolar cell. The stereocilia tufts are also myosin VIIa-positive (arrowheads). (D): Fluorescence immunochemistry showing a phalloidin-positive cell with a pear-shaped soma, and ciliary tufts (arrowheads), similar in appearance to inner hair cells. (E): Anti-prestin antibody labels the cytoplasm of a cochlear cell in culture. (F): FM1-43 labels stereocilia transduction channels, and basal and apical membrane recycling in a pear-shaped cell with a centrally located spherical nucleus. Scale bars=20 μm in (A); 10 μm in (B-F).

FIG. 4. Differentiation of olfactory neurospheres using co-culturing and supernatant treatment. (A): Co-culturing of cochlea and olfactory precursor cells resulted in myosin VIIa positive bipolar and unipolar cells with stereociliary tufts (arrowhead). (B): Diaminobenzidine immunocytochemistry showing a myosin VIIa positive cell after co-culturing olfactory neurospheres and cochlear cells for 21 days in the transwell system. The transwell system separates cells but allows diffusion of soluble factors. (C): Fluorescence immunocytochemistry showing a phalloidin positive cell with stereocilia tuft (arrowhead) after 14 days treatment of olfactory neurospheres with supernatant from cochlear cultures. (D): Double immunolabelling of a myosin VIIa and espin-positive bipolar cell after treatment of olfactory neurospheres with cochlear supernatant. Espin is co-localised with myosin VIIa in the stereocilia (arrowhead). The absence of supernatant produces myosin positive cells with no processes (inset). (E-F): Calretinin-positive pear shaped and bipolar cells with stereociliary tufts (arrowheads) observed after treatment of olfactory neurospheres with cochlear supernatant. Calretinin-positive cells are negative for the neuronal marker β-III-tubulin, which labels neurons derived from control cultures (inset). (G): Unipolar, rectangular shaped, prestin-positive cells were derived from supernatant-treated olfactory precursor cells. In the absence of supernatant, only filamentous, prestin-positive cells with multiple processes were observed (inset). Scale bars=10 μm in (A-G) and inset (G); 5 μm in insets (C), (D) and (F). Abbreviation: SN, supernatant.

FIG. 5. Quantification of hair cell-like cells. (A): The percentage of differentiated cells that formed hair cell-like cells after cochlear supernatant treatment was quantified using FM1-43 to label hair cell-like cells and PI to label total number of cells (only differentiated cells were included in total cell counts). A differentiating olfactory neurosphere contains two FM1-43 positively labelled cells (arrows). (B): After supernatant treatment, FM1-43-positive cells (arrow) were counted among total differentiated cells. (C): Without supernatant treatment, olfactory neurospheres differentiated into rare and dissimilar FM1-43-positive cells with different immunostaining (perinuclear) and morphology (multiprocess, no stereociliary tufts). (D): Summary data showing that the addition of supernatant caused a significantly greater amount of differentiation, as 2.3% of total differentiated cells were FM1-43-positive cells when treated with supernatant (22 FM1-43-positive cells of 936 PI-positive differentiated cells), whereas, in control experiments without supernatant, 0.3% of differentiated cells were FM1-43-positive-cells (3 FM1-43-positive cells of 912 PI-positive differentiated cells; *p<0.001). Scale bars=10 μm in (A-C). abbreviations; PI, propidium iodide; SN, supernatant.

FIG. 6. Surgical procedure for cell injection via mouse cochleostomy. (A): The bulla was opened to expose the lateral wall of the cochlea. Several landmarks are visible including the stapedial artery (sa), the round window (RW) and the facial nerve. The malleus (m) is shown here for demonstration purposes only as malleus exposure indicates damage to the tympanic membrane. (B): Location of injection site via cochleostomy#1 is shown posterior to the stapedial artery (sa) in the lateral wall of the cochlea. (C): Two additional injection sites via cochleostomy#2 and the round window (RW), are shown anterior to the stapedial artery (sa). (D): Schematic diagram of (C).

FIG. 7. Paint injection into whole cochleae. (A): Dissected cochleae show red paint in the spiral coils. Bones were rendered transparent with glycerol/benzol/ethanol. (B): Site of cochleostomy and paint injection in lateral wall of cochlea. Paint was evident proximal and distal to the cochleostomy. (C): Thin sections of cochleae showing paint injection into multiple cochlear compartments (arrow) and tissue plug in cochleostomy (asterisk). (D): High magnification image of boxed area in (C).

FIG. 8. Cell injection into scala media, scala tympani, scala vestibuli. (A-B): Thin sections of mouse cochleae stained with haematoxylin and eosin (H&E) showing the anatomical locations of scala vestibuli (sv), scala media (sm) and scala tympani (st) at low (A) and high magnification (B). (C): Unstained thin section of cochlea showing preservation of organ of Cord (arrow) and spiral ganglion soma (SGN). Approximate location of Reissner's membrane is indicated by dashed line. (D): Injection via cochleostomy#1 shows fluorescent cells in scala tympani and scala vestibuli. (E): Injection via cochleostomy#2 shows fluorescent cells in the scala media, the scala tympani and the scala vestibuli.

FIG. 9. Surgical treatment does not affect auditory function. (A-B): Auditory brainstem responses (ABRs) to pure tone stimuli (16 kHz) in control mice and mice after cochleostomy#1. ABR threshold determined by visual detection (arrows). Data for individual animals. (C): ABR threshold determined by signal-to-noise ratio (SNR) analysis (AxoGraphX Scientific). The sound intensity level was deemed threshold when the maximum peak amplitude was three times the standard deviation of the baseline noise. Boxes show measurement regions (inset). (D): Summary data showing that mean threshold was similar for different acoustic stimuli (click vs 16 kHz tone). The mean threshold was similar for different detection methods (visual vs SNR) for the tone stimuli and different for the click stimuli (paired t-test, p<0.05). Means±SEM.

### DETAILED DESCRIPTION OF THE PREFERRED EMBODIMENTS

The present inventors have surprisingly discovered that murine adult olfactory precursor cells can differentiate into cochlear hair cell-like cells when co-cultured with murine cochlear cells or supernatant derived therefrom.

Thus, the present invention provides a method for generating cochlear hair cell-like cells in the inner ear of a subject by administering adult olfactory precursor cells or progeny thereof to the inner ear of the subject.

The present invention also provides a method for treating a subject having or prone to an inner ear hair cell related condition, or treating a subject prophylactically to prevent or reduce the occurrence or severity of an inner ear hair cell related condition, by administering adult olfactory precursor cells or progeny thereof to the inner ear of the subject.

As used herein, the term “adult olfactory precursor cell” refers to a cell derived from the olfactory mucosa of an adult mammal, that is both self-renewing and multipotent. By “self-renewing” it is meant that the adult olfactory precursor cell is able to go through numerous cycles of cell division and maintain its undifferentiated state. By “multipotent” it is meant that the adult olfactory precursor cell can generate progeny of several distinct cell types, for example, both glial cells and neurons.

As used herein, the term “hair cell-like cell” refers to a cell that has characteristics of a hair cell, for example, immunopositive for Myosin VIIa, calretinin, phalloidin and epsin markers and immunonegative for β III tubulin marker. A hair cell-like cell may be a hair cell.

As used herein, the terms “treating” or “treatment” include administering a therapeutically effective amount of cells as defined herein sufficient to reduce or eliminate at least one symptom of the specified condition.

As used herein, the term “preventing” includes administering a therapeutically effective amount of cells as defined herein sufficient to stop or hinder the development of at least one symptom of the specified condition.

The subjects targeted for treatment by the current invention are mammals, preferably humans. The results obtained in the mouse model exemplified herein are indicative of human application. For example, human adult olfactory precursor cells have previously been isolated from neuroepithellium and cultured to form neurospheres under similar conditions to those described herein in Example 1 (see WO 03/064601). It is therefore reasonable to expect that these human neuropheres will also differentiate into human cochlear hair cell-like cells when exposed to cochlear cells or supernatant derived therefrom.

The inner ear hair cell related condition to be treated by the method of the present invention is preferably sensorineural hearing impairment. It will be appreciated that the sensorineural hearing impairment may result from inner ear cell injury, loss, or degeneration, such as that caused by a cytotoxic agent.

One advantage of the present invention is that adult olfactory neuroepithelium tissue offers an abundant and easily accessible source of adult olfactory precursor cells, a significant advantage for future autotransplantation cell therapy. Olfactory receptor neurons are exposed to the external environment and are susceptible to toxic airborne chemicals, infectious pathogens and physical damage following frontal head trauma. Hence, olfactory receptor neurons are replaced periodically throughout adult life and also have the capacity to proliferate in response to acute injury. The persistence and ability of the olfactory system to regenerate its neuroepithelium by replacing damaged or dead neurons is unique in the mammalian nervous system. Replacement and proliferation is due to the presence of multipotent stem cells in the olfactory neuroepithelium (Graziadei and Graziadei, 1979; Mackay-Sim and Kittel, 1991; Roisen et al., 2001; Ortman et al., 2003; WO 03/064601). Importantly, the ability to obtain olfactory precursor cells from neuroepithelium in the nasal cavity without permanent damage to the donor individual eliminates the need to use highly invasive and damaging procedures. Further it provides stem cells for autologous transplantation thereby reducing both the frequency and severity of rejection.

### Obtaining and Culturing Adult Olfactory Precursor Cells

Once removed from a subject, the olfactory neuroepithelium may be cultured to obtain adult olfactory precursor cells. For example, the minced neuroepithelium tissue can initially be placed into Dulbecco's modified eagle medium (DMEM) containing 1% (w/v) bovine serum albumin (BSA) (Sigma Chemicals, St Louis, USA), 50 μg/ml DNase (Sigma Chemicals, St. Louis, USA), 1 mg/ml hyaluronidase (Sigma Chemicals, St Louis, USA), 1 mg/ml collagenase (Roche, Australia) and 5 mg/ml dispase (Roche, Australia) for 1 hour at 37° C. The tissue suspension can subsequently be triturated, filtered through 150 μm wire mesh (Small Parts Inc., Miami Lakes, Fla., USA), centrifuged and resuspended in Neurobasal medium (Gibco BRL, MD, USA), containing 10% (w/v) dialysed fetal calf serum (FCS) (Gibco BRL, MD, USA), 10000 U/ml penicillin G (Sigma Chemicals, St. Louis, USA) and 20 mM glutamine (CSL, Melbourne, Australia). Cells can be filtered again through a 40 μm nylon mesh filter (BD Falcon, Franklin Lakes, Mass., USA) and collected on a 10 μm nylon mesh filter (Small Parts Inc, Miami Lakes, Fla., USA). Cultures may subsequently be grown at 37° C., 5% CO₂ in Neurobasal medium containing B27 supplement (instead of FCS), 20 ng/ml fibroblast growth factor-2 (Promega, Madison, Wis., USA), 20 ng/ml epidermal growth factor (Promega, Madison, Wis., USA), 10000 units/ml penicillin G, and 20 mM glutamine.

Other media may be appropriate, as recognised by a person skilled in the art, as well as different animal sources of sera or the use of serum free media. Furthermore, some cultures may require additional supplements, including amino acids, growth factors etc. A variety of substrata may be used to culture the cells, for example, plastic or glass, coated or uncoated substrata may be used. For example, the culture plate may be a laminin-fibronectin coated plastic plate. Alternatively, the substrata may be coated with extracellular matrix molecules (to encourage adhesion or to control cellular differentiation), collagen or poly-L-lysine (to encourage adhesion free of biological effects). The cell culture substrata may also be treated to be charged. In the case where substratum adhesion is undesired, spinner cultures may be used, wherein cells are kept in suspension. Adult olfactory precursor cells obtained from this culturing procedure may then be used for administration to the inner ear of the subject.

The present invention also relates to the use of progeny cells which are produced from in vitro culture of adult olfactory precursor cells derived from the olfactory neuroepithelium. Adult olfactory precursor cells proliferate to form tight clusters of progeny cells referred to as neurospheres after 24 hours in culture. Generally neurospheres represent a population of neural cells in different stages of maturation formed by a single, clonally expanding precursor cell. Progeny cells that make up these neurospheres are suitable for use in the present invention.

In a preferred embodiment of the invention, human nasal mucosa is cultured to obtain a neurosphere culture (see Murrell et al., 2005). For example, human nasal mucosa biopsies may initially be placed in DMEM/HAM F12 (Invitrogen, Australia) medium supplemented with 10% FCS, penicillin and streptomycin and incubated for 45 minutes at 37° C. in a 2.4 U/ml Dispase II solution (Boehringer, Germany). Laminae propriae may be separated from the epithelium under a dissection microscope with a microspatula. Sheets of olfactory epithelium may be mechanically dissociated while lamina propriae may be cut into pieces of, for example, approximately 40 μm² using a McElwain chopper (Brinkmann, Canada) and incubated in a 0.25 mg/ml collagenase H solution (Sigma, USA) for 10 minutes at 37° C. After mechanical trituration, the enzymatic activity may be stopped using a 0.5 mM ethylenediaminetetraacetic acid (EDTA) solution (Invitrogen, Australia). Cell pellets of both tissues may be resuspended in DMEM/HAM F12 culture medium containing 10% FCS plus penicillin/streptomycin and sequentially plated into flasks pre-treated with poly-L-lysine (1 μg/cm²; Sigma, USA). Eighteen hours after initial plating, floating cells and undigested pieces of epithelium and lamina propria may be transferred to other coated wells. This operation may be repeated 24 hours later.

According to the invention, the adult olfactory precursor cells or progeny thereof may be collected from culture and resuspended in a buffer before being administered to the inner ear of the subject. In one embodiment of the invention, the adult olfactory cells or progeny thereof are harvested and frozen in, for example, serum/10% DMSO prior to resuspension in the buffer. A variety of methods may be used to collect adult olfactory precursor cells or progeny thereof, including enzymatic removal (such as by trypsination), chemical methods, (e.g. cation metal chelation using EDTA or ethylene glycol-bis(β3-aminoethyl ether) NNN′N′-tetraacetic acid (EGTA), and mechanically, such as by cell scraping or in the case of cell suspension, by simple centrifugation. The “buffer” may be any suitable buffer that is generally regarded as safe and generally confers a pH from or about 4.8 to 8, preferably from or about 5 to 7. Examples include acetic acid salt buffer, which is any salt of acetic acid, including sodium acetate and potassium acetate, succinate buffer, phosphate buffer, citrate buffer, histidine buffer, or any others known in the art to have the desired effect.

Implantation of Adult Olfactory Precursor Cells into the Inner Ear

According to the method of the invention, adult olfactory precursor cells or progeny thereof are then implanted into the subject's inner ear. In the method of the present invention, adult olfactory precursor cells or progeny thereof can be implanted by any method known in the art, for example, into the scala media via basal turn penetration, into the scala tympani via round window penetration, or into the spiral ganglion via round window penetration (see Examples 7 to 9).

Generally, delivery of therapeutic agents in a controlled and effective manner with respect to tissue structures of the inner ear (for example, those portions of the ear contained within the temporal bone which is the most dense bone tissue in the entire human body) is known. Exemplary inner ear tissue structures of primary importance include but are not limited to the cochlea, the endolymphatic sac/duct, the vestibular labyrinth, and all of the compartments which include these components. Access to the foregoing inner ear tissue regions is typically achieved through a variety of structures, including but not limited to the round window membrane, the oval window/stapes footplate, and the annular ligament. The middle ear can be defined as the physiological air-containing tissue zone behind the tympanic membrane (e.g. the ear drum) and ahead of the inner ear. It should also be noted that access to the inner ear may be accomplished through the endolymphatic sac/endolymphatic duct and the otic capsule. The inner ear tissues are of minimal size, and generally accessible through microsurgical procedures.

Delivery of therapeutic cells to the inner ear of a subject can be done by contact with the inner ear as described above or through the external auditory canal and middle ear, as by injection or via catheters, or as exemplified in U.S. Pat. No. 5,476,446, which provides a multi-functional apparatus specifically designed for use in treating and/or diagnosing the inner ear of a human subject. The apparatus, which is useful in the practice of the present invention, has numerous functional capabilities including but not limited to (1) delivering therapeutic agents into the inner ear or to middle-inner ear interface tissues; (2) withdrawing fluid materials from the inner ear; (3) causing temperature, pressure and volumetric changes in the fluids/fluid chambers of the inner ear; and (4) enabling inner ear structures to be electro-physiologically monitored. In addition, other systems may be used to deliver the factors and formulations of the present invention including but not limited to an osmotic pump which is described in Kingma et al. (1992). An exemplary, commercially-available osmotic pump may be obtained from the Alza Corporation (Palo Alto, Calif., USA).

Differentiation of Adult Olfactory Precursor Cells into Cochlear Hair Cell-Like Cells In Vitro

The present invention also provides a method of generating cochlear hair cell-like cells in vitro comprising culturing adult olfactory precursor cells or progeny thereof with cochlear cells or supernatant derived therefrom, such that the olfactory precursor cells or progeny thereof differentiate into cochlear hair cell-like cells.

In one embodiment, the neurospheres can be co-cultured with cochlear cells or cochlear supernatant to differentiate into cochlear hair cell-like cells. Co-culture with cochlear cells can be performed in, for example, a 2-well chamber slide and/or a 12-well transwell culture system, where a porous 0.4 μm membrane prevents any direct contact between the adult olfactory precursor cells or progeny thereof and the cochlear cells but allows diffusion of soluble factors.

As used herein, the term “supernatant” refers to the non-cellular material produced following the in vitro culturing of cochlear cells. Typically, the supernatant is produced by culturing the cells in the medium under suitable conditions and time, followed by removing the cellular material by a process such as centrifugation or filtration. The supernatant may or may not have been subjected to further purification steps before administration. In a preferred embodiment, the supernatant comprises no live cells.

In one embodiment, the harvested cochlear supernatant is frozen. Prior to use, the cochlear supernatant is filtered through, for example, a 40 μm mesh or centrifuged to remove debris, then added undiluted to the olfactory neurosphere cultures.

Cochlear cells used in the culture method of the present invention can be obtained by any suitable method. For example, primary cochlear hair cells can be obtained by standard enzymatic digestion of the neuroepithelium and cell filtration techniques. Cultures can subsequently be grown at 37° C., 5% CO₂ in Neurobasal medium containing B27 supplement. Cell lines such as, OC-1 and OC-2 may also be used as a source of cochlear hair cells for use in the method of the invention.

The cochlear hair cell-like cells differentiated from the adult olfactory precursor cells or progeny thereof may be characterised by morphology, immunochemistry, flow cytometry and immunohistochemistry, the methods of which are known to those skilled in the art. Preferably the cochlear hair cell-like cells are characterised as Myosin VIIa, calretinin, phalloidin and epsin positive and β III tubulin negative. The cochlear hair cell-like cells may also be characterised electro-physiologically as described in Kros et al. (1990) and Kros et al. (1998).

Cochlear hair cell-like cells obtained by this method of culture can then be administered directly to a subject by the transplantation techniques described above. It will be understood therefore, that the cochlear hair cell-like cells obtained by this method of culture can be used to treat or prevent an inner hair cell related condition.

### Testing for Diagnosis and Treatment of Hearing Impairment

Tests are known and available for diagnosing hearing impairments. One of the most commonly employed hearing tests is pure tone audiometry that involves measuring the threshold of hearing for pure tones of normally audible frequencies generally varying from 200 to 8000 Hertz. Comparison of pure tone testing of threshold by air (sounds that reach the inner ear through the ear canal) and bone conduction (sounds transmitted through bones) enables discrimination between sensorineural and conductive hearing loss. Speech discrimination tests that measure a person's ability to identify words can also be used as an indicator of sensorineural hearing loss. The test includes presentation of about 50 selected monosyllabic words at an easily detectable intensity level. The speech discrimination score is the percentage of words correctly identified. A Tympanometry test that creates variations of air pressure in the ear canal enables the condition of the middle ear and mobility of the eardrum to be examined. Other hearing tests may be employed and would be familiar to those skilled in the art.

The effectiveness of treating hearing impairments with the methods of the invention can be evaluated by the following signs of recovery, including recovery of normal hearing function, which can be assessed by known diagnostic techniques including those discussed herein, and normalization of nerve conduction velocity, which is assessed electro-physiologically.

The references cited throughout the specification are herein incorporated in their entirety. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The invention herein after described by way of the following non-limiting Examples and with reference to the accompanying figures.

### EXAMPLES

### Example 1

### Materials and Methods

### Primary Adult Olfactory Precursor Cells

To isolate olfactory precursor cells, olfactory turbinates were dissected from 10-15 adult CBA/CaH mice aged 6 weeks. Mice were anaesthetised with CO₂, decapitated and olfactory turbinates were removed and placed in DMEM containing 9.6 mg/ml HEPES buffer. Tissue was centrifuged and supernatant was removed before placing minced tissue into DMEM containing 1% (w/v) BSA (Sigma Chemicals, St Louis, USA), 50 μg/ml DNase (Sigma Chemicals, St. Louis, USA), 1 mg/ml hyaluronidase (Sigma Chemicals, St. Louis, USA), 1 mg/ml collagenase (Roche, Australia) and 5 mg/ml dispase (Roche, Australia) for 1 hour at 37° C. Tissue suspension was triturated, filtered through a 150 μm wire mesh (Small Parts Inc., Miami, Fla., USA), centrifuged and resuspended in Neurobasal medium (Gibco BRL, MD, USA), containing 10% (w/v) dialysed FCS (Gibco BRL, MD, USA), 10000 U/ml Penicillin G (Sigma Chemicals, St. Louis, USA) and 20 mM glutamine (CSL, Melbourne, Australia). Cells were filtered twice more through a 40 μm nylon mesh filter (BD Falcon, Franklin Lakes, Mass., USA) for size exclusion and subsequently the olfactory precursors were collected on a 10 μm nylon mesh filter (Small Parts Inc, Miami, Fla., USA). Cultures were grown at 1×10⁵ cells/ml at 37° C. in 5% CO₂ in Neurobasal medium or DMEM/F12 (1:1) containing B27 supplement (instead of FCS), 20 ng/ml fibroblast growth factor-2 (Promega, Madison, Wis., USA) and 20 ng/ml epidermal growth factor (Promega, Madison, Wis., USA), 10000 units/ml penicillin G, and 20 mM glutamine. During normal growth conditions the medium was not changed for the duration of the experiments.

### Bromodeoxyuridine Incorporation Assay

The olfactory neurospheres were incubated with bromodeoxyuridine (BrdU) labelling reagent (Sigma Chemicals, St Louis, USA) that was added to the growth medium at a final concentration of 10 μM upon plating. Cells were grown in Labtek 4 well chamber slides (Nunc, Rochester, N.Y., USA) for 5 days at 37° C., 5% CO₂. The cells were fixed in 70% ethanol (in 50 mM glycine buffer) for 20 minutes at −20° C., washed in phosphate-buffered saline (PBS) and permeabilized with 0.3% TritonX-100 for 15 minutes at room temperature. Cells were incubated with monoclonal anti-BrdU antibody (1:10; Chemicon, Temecula, Calif., USA) for 1 hour at room temperature and detected using goat anti-mouse immunoglobulin G (IgG) conjugated Alexa Fluor® 488 antibody (1:50; Promega, Madison, Wis., USA) for 30 minutes at room temperature. Cells were washed in PBS and mounted in Vectashield (Vector Laboratories, Burlingame, Calif., USA) mounting medium. Fluorescence was visualised using a laser scanning confocal microscope (Leica, Heerbrugg, Switzerland).

### Immunolabelling

Hair cells in the intact organ of Corti were characterised by immunohistochemistry using several hair cell specific markers (see Table 1). Adult CBA/CaH mice aged 6 weeks (n=8) were given an overdose of intraperitoneal Euthal (80 mg/kg) and transcardially perfused with ice-cold normal saline followed by 4% paraformaldehyde (PFA). Cochlear tissue was dissected and processed for paraffin embedding. Cross sections (6 μm thick) were collected onto electrostatic glass slides (Menzel-Glaser, Braunschweig, Germany). Sections were dewaxed in Histoclear (National Diagnostics, Atlanta, Ga., USA) and rehydrated through a graded series of alcohols. The cochlear tissue used for anti-myosin VIIa immunohistochemistry was immersion fixed overnight in formalin, cryoprotected and subsequently sectioned (14 μm thick) on a cryostat (Leica, Heerbrugg, Switzerland) and permeabilised with Triton 0.3%.

Non-specific staining was blocked in 10% serum (of the same species as the secondary antibody) and 1% BSA in PBS for 1 hour at room temperature. This was followed by incubation with the primary antibody diluted in PBS (see Table 1) for 1 hour at room temperature. Control sections were incubated with 1% normal serum (of the same species as the secondary antibody) in PBS and processed in parallel.

For immunoperoxidase studies, sections were washed in PBS, submerged in 0.3% hydrogen peroxide for 15 minutes at room temperature. Following further washes in PBS, sections were incubated with either biotinylated goat anti-rabbit IgG, rabbit anti-goat IgG or horse anti-mouse IgG secondary antibody (Vector Laboratories, Burlingame, Calif., USA) diluted 1:300 in PBS for 30 minutes at room temperature. In order to amplify specific binding, a Vectastain® ABC kit (Vector Laboratories, USA) was used according to manufacturer's instructions. For detection of specific binding, a chromagen diaminobenzidine (DAB) kit (DakoCytomation, USA) was used. The Dab solution was applied for 2-3 minutes before sections were washed in PBS and coverslipped using aquamount (BDH, Merck, Darmstadt, Germany).

For fluorescent immunohistochemistry studies, sections were incubated with either goat anti-rabbit IgG conjugated Alexa Fluor® 488, donkey anti-goat IgG conjugated Alexa Fluor® 488 or goat anti-mouse IgG conjugated Alexa Fluor® 488 secondary antibody diluted 1:50 in PBS for 30 minutes at room temperature.

Double-labelling immunocytochemistry was performed using two primary antibody combinations: 1) anti-calretinin (rabbit) and anti-prestin (goat) and 2) anti-calretinin (rabbit) and anti-β III tubulin (mouse). Sections were incubated with primary antibodies for 1 hour at room temperature. For the primary antibody incubation of calretinin and prestin, the primary antibodies were incubated sequentially. Sections were subsequently incubated with 1) donkey anti-rabbit IgG conjugated Alexa Fluor® 594 (Promega, Madison, Wis., USA) and donkey anti-goat IgG conjugated Alexa Fluor® 488 (Promega, Madison, Wis., USA), or 2) goat anti-rabbit IgG conjugated Alexa Fluor® 594 (Promega, Madison, Wis., USA) and goat anti-mouse IgG conjugated Alexa Fluor® 488 (Promega, Madison, Wis., USA) secondary antibodies diluted 1:50 in PBS for 30 minutes at room temperature. Slides were mounted in Vectashield (Vector Laboratories, Burlingame, Calif., USA) and images were captured using a Leica DC480 (Leica, Heerbrugg, Switzerland) digital camera attached to a Zeiss Axiophot microscope (Carl Zeiss, Germany).

Hair cells in culture were characterised by immunocytochemistry with a similar range of hair cell-specific markers used in the intact organ of Corti. After 7-14 days in culture, cells (cochlear or differentiated precursor cells) were rinsed in PBS and fixed in 2% PFA (ProSciTech, Thuringowa, Australia). Following fixation, cells were permeabilized in 0.1% Triton X-100, rinsed in PBS, blocked in 10% normal serum (from the same species as the secondary antibody) in PBS, and then incubated with primary antibodies diluted in PBS containing 1% BSA (see Table 1). Following three washes in PBS, the cells were incubated with the secondary fluorescent antibodies diluted 1:300 in PBS for 30 minutes in the dark. Unless otherwise stated all incubations were at room temperature in a humidified chamber. Slides were mounted in Vectashield (Vector Laboratories, Burlingame, Calif., USA) and images were captured using a Leica DC480 digital camera (Leica, Heerbrugg, Switzerland) attached to a Zeiss Axiophot microscope (Carl Zeiss, Germany).

TABLE 1
Cell type specific markers, their optimal concentrations and source.

| | Cell type and | | | |
|---|---|---|---|---|
| Marker | distribution | Dilution | Antibody type | Source |
| Myosin VIIa | Hair cell soma and | 1:100-200 | Rabbit polyclonal | Novus Biologicals, |
| | stereocilia | | | USA |
| Calretinin | Inner hair cell | 1:1400 | Rabbit polyclonal | Swant, |
| | soma and | | | Switzerland |
| | stereocilia | | | |
| Prestin | Outer hair cell | 1:80 | Goat polyclonal | Santa Cruz, USA |
| | soma | | | |
| Math1 | Hair cell nuclei | 1:50-100 | Rabbit polyclonal | Abcam, UK |
| Espin | Stereocilia | 1:50 | Mouse | BD Transduction |
| | | | monoclonal | Laboratories, USA |
| Phalloidin | Hair cell soma and | 1:50 | Labelled | Promega, USA |
| | stereocilia | | secondary | |
| FM1-43 | Hair cell soma and | 5 μg/ml | Styryl dye | Promega, USA |
| | stereocilia | | | |
| β III tubulin | Neuronal soma | 1:100 | Mouse | Sigma, USA |
| | and neurites | | monoclonal | |
| Keratin | Epithelial cells | 1:400 | Rabbit polyclonal | Dako, USA |
| Pax-6 | Sustentacular | 1:200 | Rabbit polyclonal | Chemicon, USA |
| | cells, olfactory | | | |
| | glandular cells | | | |
| Glial fibrillary | Astrocytes and | 1:800 | Rabbit polyclonal | Dako, USA |
| associated protein | other glial cells | | | |
| (GFAP) | | | | |

### Primary Adult Cochlear Cultures

To characterise hair cells in vitro, primary cultures were prepared from cochlea dissected from 3-4 adult CBA/CaH mice aged 6 weeks. Although every effort was made to limit the dissection to cochlear tissue, the inclusion of some vestibular tissue cannot be ruled out. Mice were anaesthetised with CO₂, and cochleae were removed and placed in DMEM (Gibco BRL, MD, USA) containing 9.6 mg/ml HEPES buffer. The protocol of enzymatic digestion and cell filtration is the same as for primary olfactory neurosphere cultures. Cells were filtered through a 40 μm nylon mesh filter (BD Falcon, Franklin Lakes, Mass., USA) prior to plating into Labtek tissue culture chamber slides (Nunc, Rochester, N.Y., USA) or transwell 12 well plates (Costar Corning, Mass., USA), Cultures were grown at 1.25×10⁶ cells/ml at 37° C. in 5% CO₂ in Neurobasal medium containing B27 supplement instead of FCS.

### Differentiation of Adult Olfactory Precursor Cells in Culture

After 7 days in culture, precursor cells were differentiated into hair cell-like cells by withdrawal of growth factors and either co-culturing with primary cochlear cultures or exposure to cochlear supernatant. Co-culturing of primary olfactory neurosphere cultures with primary cochlear cultures was performed in 2-well chamber slides and in the 12-well transwell culture system where a porous 0.4 μm membrane prevents any direct contact between olfactory and cochlear cells but allows diffusion of soluble factors. Tissue culture supernatant was collected from cochlear cultures (1-21 days in vitro), and used immediately or frozen for future use. Prior to use it was centrifuged and filtered through a 40 μm mesh to remove debris, then added undiluted to olfactory neurosphere cultures. Control olfactory neurosphere cultures were allowed to differentiate in the absence of supernatant and growth factors.

Quantification of Differentiation of Olfactory Neurospheres into Hair Cell-Like Cells

To quantify the extent to which the olfactory neurospheres differentiated into hair cell-like cells, FM1-43-positive cells (hair cell marker) were counted in the presence and absence of cochlear supernatant. Neurospheres were treated with and without supernatant, allowed to differentiate for 11 days, then exposed to FM1-43FX for 1 minute, washed thoroughly in PBS, fixed in 2% PFA, permeabilised (0.1% Triton X-100) and then labelled with propidium iodide (10 μg/ml) (Sigma Chemicals, St. Louis, USA). Cells were examined using a Zeiss Axiophot microscope (Carl Zeiss, Germany). Fields were chosen at random (n=27 with supernatant treatment; n=22 without supernatant treatment) and the number of FM1-43-positive cells was counted and calculated as a percentage of the total number of propidium iodide-positive differentiated cells.

### Experimental Animals and Surgical Procedures

A total of 23 CBA/CaH mice aged 18-42 postnatal days were used in this study. Preliminary surgeries were performed on mouse cadavers sacrificed by cervical dislocation or ketamine overdose (n=19). Some animals were pre-perfused with 4% paraformaldehyde to improve anatomical preservation (n=12). For minimal trauma surgery, mice were anaesthetised with 75 mg/kg ketamine (Cenvet Australia, Maryong, NSW, Australia) and 15 mg/kg xylazine (Provet, Castle Hill, NSW, Australia) (n=4). Mice were placed in a supine position with the neck extended. A minimally invasive procedure was performed to access the middle and inner ear. The surgical procedure was completed in 40 minutes and performed under an Olympus dissecting microscope with 0.63-4× magnification. Photographs were taken with an Olympus C-7070 wide zoom digital camera attached to the dissecting microscope.

Cell Injection into the Cochlea

Green fluorescent cells were used to visualise the spatial distribution of injected cells. MCF10A immortalised human mammary epithelial cells (Soule et al., 1990) expressing the ecotropic retroviral receptor (MCF10A^{EcoR} cells, a kind gift of Drs. Danielle Lynch and Joan Brugge) were infected with pSIREN-retroQ-ZsGreen (Clontech, Mountain View, Calif., USA) and cultured as previously described (Debnath et al., 2003). In brief, the ecotropic packing cell line Phoenix-Eco was transfected with 10 μg of plasmid DNA using PolyFect (Qiagen, Doncaster, VIC, Australia). Retroviral supernatants were used to overlay a subconfluent culture of MCF10A^{EcoR} cells. Green fluorescent-positive cells were then sorted to homogeneity using flow cytometry. Prior to injection, cells were harvested with trypsin-ethylenediaminetetraacetic acid (EDTA, Invitrogen, Australia) and resuspended in phosphate buffered saline/EDTA at a concentration of 4×10⁶ cells/ml.

Cell injections were made using a Hamilton syringe and a glass capillary needle, broken to a tip diameter of 100 μm and inserted into the cochleostomy. A total volume of 1 μl of fluorescent cells was injected over 1 minute. In some cases, the cell suspension was substituted with red paint to visualise the cochlear access provided by the injection site. The red paint was diluted with methyl salicylate to facilitate expulsion from the injection syringe and movement throughout the cochlea. The injected cells or paint remained in the cochlea for approximately 10 minutes before the cochlea were removed from the animal and prepared for whole mounts or histological analysis in thin sections.

### Preparation of Cochlear Whole Mounts

Following injection, cochleae were prepared as previously described (Pau et al., 2005), fixed in Bodian's fixative (75% ethanol, 5% formalin, 5% glacial acetic acid) for 60-120 minutes, washed in distilled water for 30 minutes, and placed in 70% ethanol for 24 hours. Specimens were placed in 3% potassium hydroxide for 4 days to remove soft tissues, transferred to a solution containing glycerol, 70% ethanol and benzol (in the ratio of 2:2:1) to render bones transparent, and stored in glycerol and 70% ethanol (in the ratio of 1:1). Whole cochleae were examined with an Olympus dissecting microscope and photographed with an Olympus C-7070 camera.

### Preparation of Cochlear Thin Sections

Cochlea were fixed in 4% paraformaldehyde for one day, decalcified in 10% EDTA for one day, then placed in 20% sucrose in phosphate buffered saline for 3 days. Cochleae were then transferred to optimal cutting temperature (OCT, Tissue-Tek) medium and placed under gentle vacuum for 10 minutes prior to freezing and thin sectioning on a cryostat. Adjacent thin sections (25 μm thick) were mounted on slides and viewed under normal or fluorescence microscopy. Some sections were stained with haematoxylin and eosin (H&E) for anatomical observation. Images were captured using a Leica DC480 digital camera (Leica, Wetzlar, Germany) attached to a Zeiss Axiophot microscope (Carl Zeiss, North Ryde, NSW, Australia).

### Testing Auditory Function

Auditory function was assessed by measuring auditory brainstem response (ABRs) thresholds in the left ear of 21-32 week old mice anaesthetised with ketamine and xylazine (n=10). ABR thresholds were determined in control mice (n=7) and in mice 3-4 months after cochleostomy (n=3), with (n=2) or without (n=1) saline injection. Subdermal needle electrodes were positioned at the vertex (active), lateral to the left cheek (reference) and at the base of the back (ground). Electrodes were adjusted to minimize impedance (<5 KOhm). Acoustic stimuli were delivered via an electrostatic insert phone fitted into the animal external ear canal (Tucker Davis Technologies) and held in place with a micromanipulator in a foam-padded, shielded acoustic chamber. The non-tested ear was blocked with a foam earplug in the ear canal. The output was calibrated with a calibration kit (Tucker Davis Technologies), which included a MA3 microphone amplifier, SigCal software and a quarter inch calibrated microphone (AcoPacific). Calibrations were made with reference to the programmed output at 90 dB SPL.

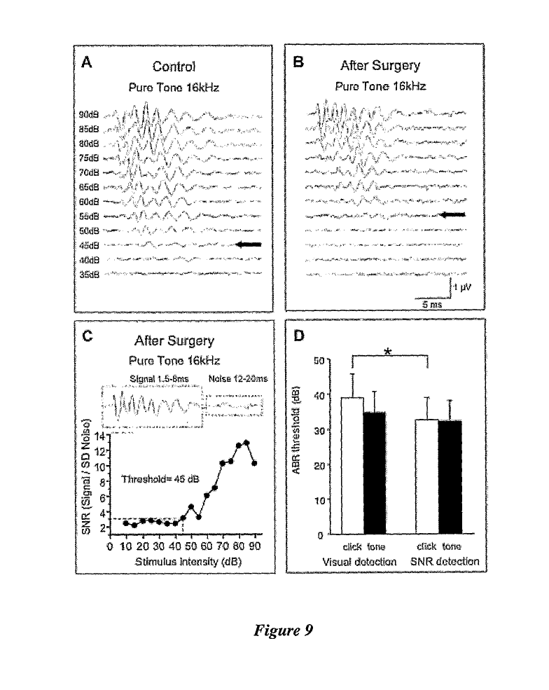

Clicks (1 ms duration) and tone pips (16 kHz; 1 ms rise/fall; 3 ms duration) were delivered and ABRs were obtained by reducing the intensity in 5 dB steps beginning at 90 dB. Threshold detection was determined by two methods: 1) visual detection and 2) signal-to-noise ratio detection. Visual detection was performed by independent observers blind to the experimental conditions. The signal-to-noise ratio was determined by comparing the absolute amplitude of the maximum peak occurring within 1.5-8 ms latency from the onset of stimuli to the standard deviation of the baseline noise over 12-20 ms latency from the onset of stimuli (AxoGraphX Scientific Software). The sound intensity level was deemed threshold when the maximum peak amplitude was three times the standard deviation of the baseline noise (see FIG. 9C). Statistics are quoted as mean±standard error of the mean (SEM). The F statistic was used to confirm equality of variances between populations before applying a two-tailed paired Student's t-test (Statview, SAS Institute, US).

### Example 2

### Characterisation of Primary Olfactory Precursor Cells

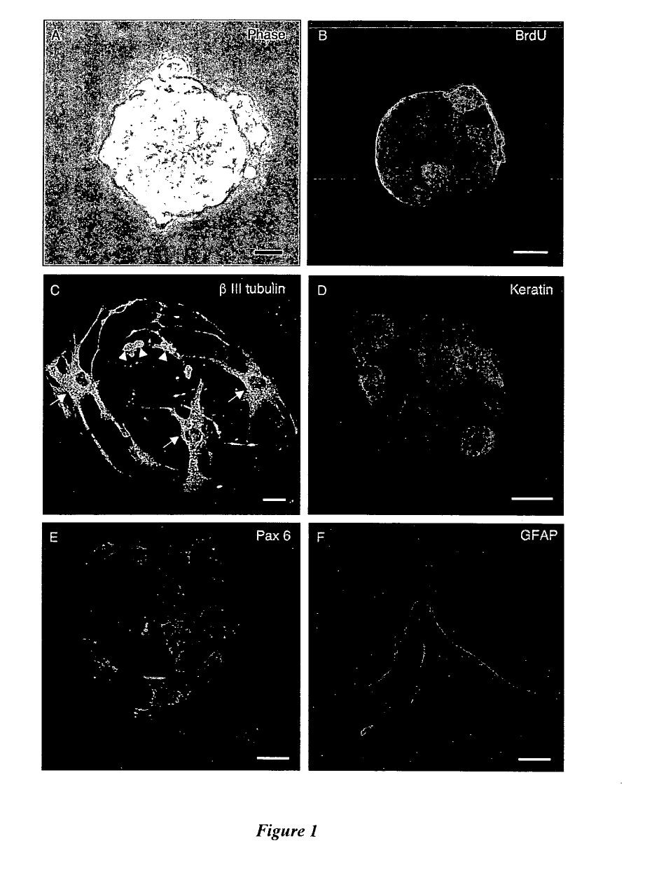

Olfactory precursor cells were prepared from dissociated primary cell cultures (n=16) of adult mouse olfactory turbinates. Precursor cells proliferated to form tight clusters of cells (neurospheres) after 24 hours in culture. Neurospheres were typically 100-200 μm in diameter, but could reach 500 μm in diameter (see FIG. 1A). Single round cells radiating from the neurospheres were commonly seen (see FIG. 1A). Some cells within the neurospheres were immunopositive for the BrdU antibody, a marker of the S-phase of the cell-cycle (see FIG. 1B). Trituration of neurospheres and single cell disposition experiments using fluorescence-activated cell sorting (FACS Calibur, BD Falcon, Franklin Lakes, Mass., USA) gave rise to secondary neurospheres, which provides additional evidence that neurospheres were self-replicating (data not shown). Upon removal of growth factors, olfactory neurospheres spontaneously differentiated into cells showing positive immunoreactivity for the neuronal antibody marker (β III tubulin), the epithelial cell marker (keratin), the sustentacular cell/glandular cell marker (Pax-6) or the glial cell marker (glial fibrillary associated protein, GFAP) (see FIGS. 1C-F). In summary, olfactory precursor cells were able to form spheres, showed proliferative capacity and were able to differentiate into neuronal and non-neuronal cell types.

### Example 3

### Characterisation of Hair Cells in the Organ of Corti

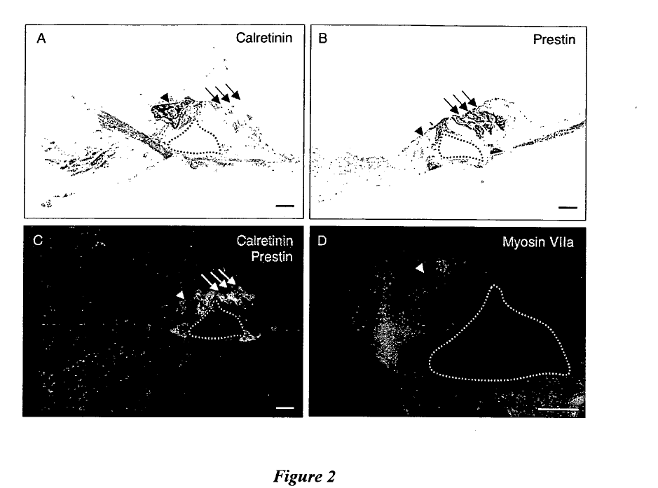

Hair cells in the adult organ of Corti (cochlea, n=9) were analysed in thin sections of cochlea using markers of inner and outer hair cells. Inner hair cells and spiral ganglion nerve fibres showed positive immunoreactivity to the calcium binding protein, calretinin (see FIG. 2A). No staining was observed in outer hair cells with the calretinin antibody. Outer hair cells were positively labelled with prestin, a transmembrane motor protein present in these cells (see FIG. 2B). Double labelling immunofluorescence shows there was no co-localisation of calretinin and prestin in the adult mouse organ of Corti, confirming the specificity of these antibody markers (see FIG. 2C). Myosin VIIa, a motor protein essential to sensory epithelia, labelled the cytoplasm of the inner hair cell shown in FIG. 2D.

### Example 4

### Characterisation of Hair Cells in Culture

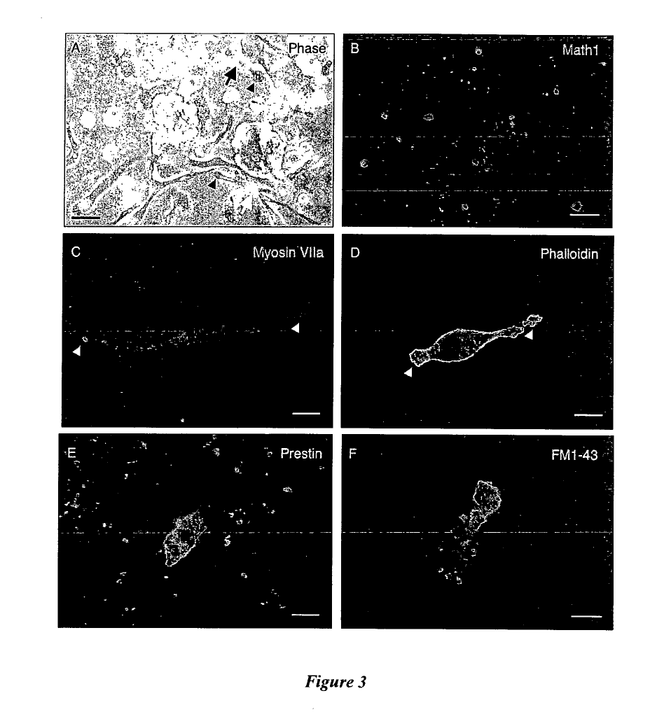

Dissociated primary cell cultures (n=11) from adult cochlea were characterised according to morphology and immunocytochemistry. Round immature cells, alone or in clusters, were observed on initial plating of primary mouse cochlear cultures. After 7-14 days in vitro, cells developed into two different types: 1) pear-shaped cells with ciliary tufts, and centrally located round nuclei, and 2) bipolar cells with oval soma and distal tufts (see FIG. 3A). Several different fluorescent antibody markers were used to characterise cochlear cells in culture. Antibodies were specific for hair cells and some were additionally specific for inner versus outer hair cells. The Math1 antibody labelled the nuclei of hair cells that appeared in the cochlear cultures 6 days post-plating (see FIG. 3B). The stereocilia and cytoplasm of pear-shaped cells with bipolar processes labelled positively with the myosin VIIa antibody (see FIG. 3C). Phalloidin labelled the extracellular rim and stereocilia of a pear-shaped cell with bipolar ciliary tufts (see FIG. 3D). The antibody for prestin showed cytoplasmic immunoreactivity in a cell where no processes or ciliary tufts were detected (see FIG. 3E). In addition, hair cells were labelled with FM1-43, a vital fluorescent dye that enters living hair cells either via transduction channels on the stereocilia, or through membrane recycling at the apex of inner and outer hair cells, or the base of inner hair cells. Fluorescent labelling for FM1-43 was observed in a pear-shaped cell with a unipolar tuft (see FIG. 3F).

### Example 5

### Differentiation of Adult Precursor Cells into Hair Cell-Like Cells

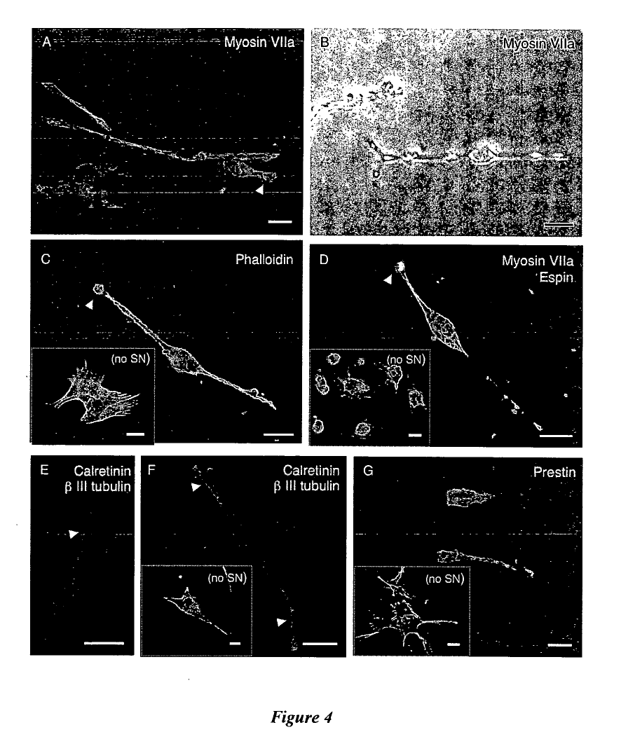

Differentiation of precursor cells was observed when cells were co-cultured with primary cochlear cultures (experiments, n=7), or exposed to cochlear supernatant (experiments, n=8). Initially, precursor cells and cochlear cultures were co-cultured in direct contact (experiments, n=3). Immunocytochemistry using anti-myosin VIIa showed positively labelled pear-shaped cells and bipolar cells (see FIG. 4A). Subsequently, co-culturing using a transwell system (experiments, n=4) was used to prevent direct contact of precursor and cochlear cells while allowing diffusion of soluble factors. As a control, olfactory precursor cells were co-cultured with medium alone in the transwell system and did not differentiate. When co-cultured with cochlear cells in the transwell, precursor cells were transformed from round, immature cells with no processes to cells with unipolar or bipolar processes following 7-14 days in culture. The differentiated precursor cells possessed a different morphology to olfactory neurons in culture (personal observations, Graziadei and Graziadei (1979). In all transwell experiments, adult precursor cells differentiated into myosin VIIa-positive cells, suggesting that a diffusible factor may be able to induce differentiation of adult precursor cells into hair cell-like cells (see FIG. 4B).

The presence of a diffusible differentiation factor was investigated further by supernatant treatment. The supernatant from cochlear cultures was collected, filtered and added to olfactory precursor cell cultures while control cultures were allowed to differentiate in the absence of cochlear supernatant. After 7-14 days of treatment, the precursor cells differentiated into bipolar cells whose soma and stereocilia were positively labelled for phalloidin, myosin VIIa, espin, calretinin and prestin (see FIG. 4C-G). Using the same antibody markers, the cells in the control cultures with no cochlear supernatant treatment show different morphologies to the unipolar or bipolar hair cell-like cells (inset 4C-G). In double labelling experiments, the bipolar cells labelled positively for calretinin (inner hair cell marker), and negatively for β III tubulin (neuronal marker), suggesting the bipolar cells were not spiral ganglion neurons (FIGS. 4E and F).

### Example 6

### Quantification of Differentiated Olfactory Precursors into Hair Cell-Like Cells

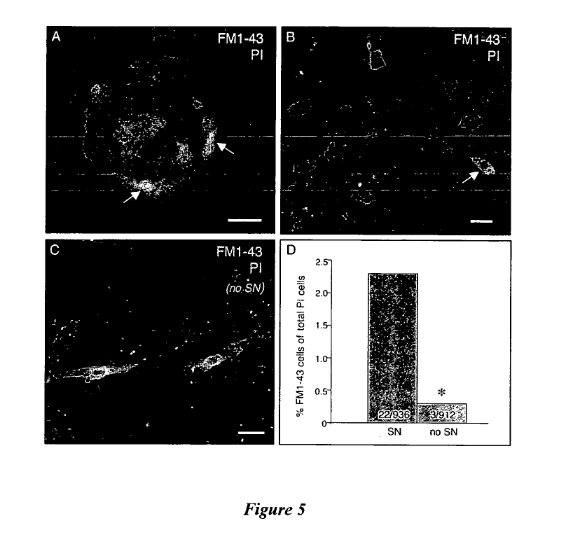

The number of hair cell-like cells produced by addition of cochlear supernatant to olfactory precursor cells was quantitated using FM1-43 to label hair cell-like cells and propidium iodide to determine the total number of cells. Microscopic analysis and quantification in random fields showed that most cells had differentiated and a small population of cells were FM1-43-positive (see FIGS. 5A and B). Some of the FM1-43-positive cells seemed to be radiating directly from the remnants of an olfactory neurosphere (see FIG. 5A). When no cochlear supernatant was added, but precursor cells were allowed to differentiate, a significantly reduced number of cells were FM1-43-positive (see FIG. 5C). These cells showed perinuclear localisation of FM1-43 and had different morphology from that of FM1-43 cells observed in the presence of supernatant. Summary data show that the addition of supernatant caused a significantly greater amount of differentiation (p<0.001, χ² test), as 2.3% (22 of 936 cells) of total differentiated cells were FM1-43-positive cells after cochlear supernatant treatment. In contrast, 0.3% (3 of 912 cells) were FM1-43-positive without supernatant treatment (see FIG. 5D).

### Example 7

### Microsurgery for Mouse Cochleostomy

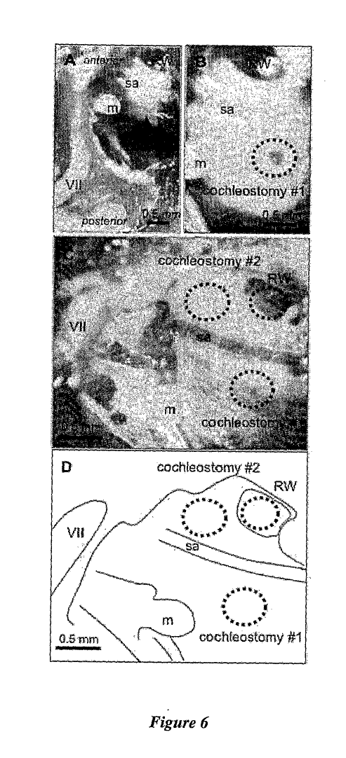

Minimal trauma microsurgery was initiated following ketamine/xylazine anaesthesia. The level of anaesthesia was monitored with corneal and withdrawal reflexes, and additional anaesthetic was applied at half dose if necessary. A postauricular incision was made and the flap retracted anteriorly. The facial nerve was preserved in its position posterior to the external auditory canal (see FIG. 6A). The muscle overlying the bulla was divided with microscissors and electrocautery was applied to prevent bleeding. The minimally invasive technique was aided by the use of 3/0 silk sutures to retract the adjacent soft tissue and increase the surgical access. The bulla was opened by sharp dissection or by cutting and/or diamond burrs (0.5 and 1.0 mm) to reduce the degree of bony trauma to the bulla and cochlea. Middle ear landmarks including the stapedial artery and round window were identified and served as orientation landmarks for the cochleostomy (see FIG. 6A). Cauterisation of the stapedial artery was performed in a limited number of animals (n=2) using a specialised tool for small animal microsurgery which enabled a localized and brief cauterisation (I-Stat, Medtronic). Cauterization of the stapedial artery did not affect animal survival.

The cochleostomy was made with a fine gauge needle (29G) from the middle ear cavity into the cochlea. A fat and muscle plug was used to seal the cochleostomy site following cell injection. A single dose of analgesic (subcutaneous ketoprofen, 1 mg/kg) was administered prior to surgery and then daily for 3 days post-surgery. Fluid therapy (subcutaneous saline, 10% blood volume) was administered pre and post-surgery. Animals showed full recovery within 4 hours.

### Example 8

### Injection into Scala Media, Scala Tympani and Scala Vestibuli

Cells were injected into the cochlea via three injection sites: cochleostomy #1, cochleostomy #2 and the round window (see FIG. 6C). Cochleostomy site #1 was posterior to the stapedial artery (see FIG. 6B) while cochleostomy site #2 was immediately anterior to the stapedial artery midway between the oval and round windows (see FIG. 6C). Cochleostomy site #2 was chosen after comprehensive drilling on dissected cochlea suggested that cochleostomy site #2 could provide direct access to scala media (data not shown). However space constraints of cochleostomy #2 caused by the proximity of the stapedial artery and the round window precluded future use of this injection site.

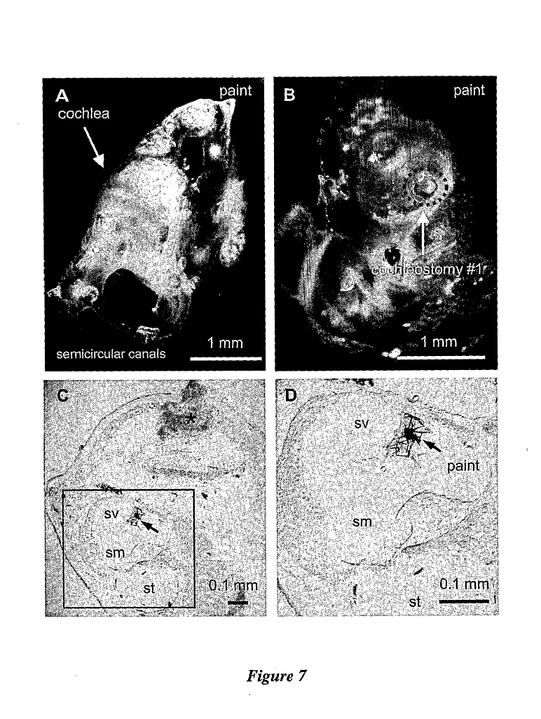

The pipette was advanced through the membranous wall of the cochlea or the round window in an anterosuperior direction. In the first instance, red paint was injected to confirm access to the cochlea (see FIG. 7A-D). Red labelling was observed in multiple spiral turns of whole cochleae and was present proximal and distal to the site of the cochleostomy (see FIGS. 7A and B). No labelling was observed in the vestibular ducts. Histological analysis of thin-sections of cochleae also revealed red paint crystals in the scala vestibuli and the scala tympani (FIGS. 7C and D).

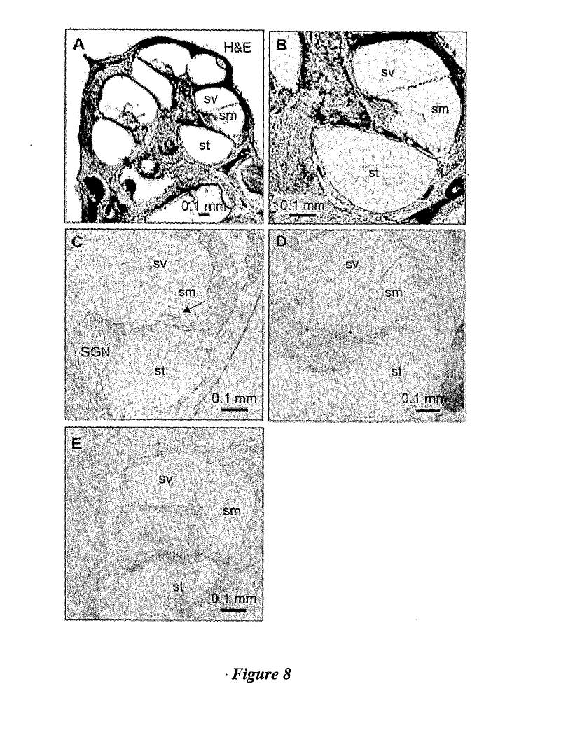

Green fluorescent cells (ZsGreen-MCF10A) were injected into the cochlea and their distribution was analysed in thin sections with fluorescence microscopy (n=12; FIG. 8). Control cochleae were sectioned and stained with H&E to establish normal cochlear morphology at successive spirals (see FIGS. 8A and B). Cochlear tissues were preserved following cell injection with stria vascularis, organ of Corti, and spiral ganglion soma still intact (see FIG. 8C). Green fluorescent cells injected at cochleostomy site #1 were observed in the scala vestibuli and the scala tympani (n=3; see FIG. 8D). Cells injected at cochleostomy site #2 were observed throughout the cochlea including the scala media, the scala vestibuli and the scala tympani (n=2; see FIG. 8E). Cells injected via the round window were observed in the scala tympani (n=1; see FIG. 8E). In some cases, fluorescence could not be detected in the cochlear compartments regardless of the cell injection site (n=6).

### Example 9

### Maintenance of Auditory Function

To determine if the microsurgical technique affected auditory function, the auditory brainstem response was recorded in 21-32 week old mice (n=11) following ketamine/xylazine anaesthesia. The ABRs were compared between control mice (n=7) and in mice receiving a cochleostomy #1 (n=3; see FIG. 9). The ABR was evoked by two types of stimuli (click and 16 kHz tone). ABR thresholds were detected by visual inspection and by computerized signal-to-noise ratio detection (see FIG. 9A-C). In control mice, visual inspection yielded a mean ABR threshold that was similar for click (39±7 dB) and tone stimuli (35±6 dB). The signal-to-noise ratio detection method yielded a mean ABR threshold that was similar for the click (33±6 dB) and tone stimuli (33±6 dB). Thus the threshold level was similar for different acoustic stimuli (click or tone) as confirmed by two independent detection methods.

A comparison of the detection methods revealed that the signal-to-noise ratio detection method estimated similar threshold levels for the tone stimuli and lower threshold levels for the click stimuli (p<0.05; paired t-test; see FIG. 9D). In mice receiving a cochleostomy (n=6), animals were deceased due to blood loss during surgery (n=1), anesthetic complications during ABR testing (n=1), and post-operative circling (n=1). The mean ABR threshold for combined click and tone stimuli was 15 dB greater after surgery (control 32.7±4.2 dB, surgery 48±3 dB) when the mouse had received a small sized cochleostomy (≦0.4 mm) and was sibling matched to control mice. Threshold levels after surgery were equal or lower than threshold levels in control mice in 20% of auditory tests. In mice that were 10 weeks older than control mice and had received a large sized cochleostomy (0.8-1 mm; n=2), the ABR threshold was 55 dB greater after surgery for combined click and tone stimuli (77:5±2.5 dB) compared to control mice.

### REFERENCES

Debnath et al. (2003) Methods 30:256-268.
Forge and Nevill (1998) J Comp Neurol 397:69-88.
Gillespie and Shepherd (2005) Eur J Neurosci 22:2123-2133.
Graziadei and Graziadei (1979) J Neurocytol 8:1-18.
Gritti et al. (1996) J. Neurosci 16: 1091-1100.
Hu et al. (2005) Exp Cell Res 302:40-47.
Izumikawa et al. (2005) Nat Med 11:271-276.
Kawamoto et al. (2003a) Mol Ther 7:484-492.
Kawamoto et al. (2003b) J Neurosci 23:4395-4400.
Kawamoto et al. (2004) Mol Ther 9:173-81.
Kingma et al. (1992) J Neurosci Met 45:127-134.
Kros et al. (1990) J Physiol 421: 263-291.
Kros et al. (1998) Nature 16:281-284
Kukekov et al. (1999) Exp Neurol 156: 333-344.
Li et al. (2003a) Proc Natl Acad Sci USA 100:13495-13500.
Li et al. (2003b) Nat Med 9:1293-1299.
Lopez et al. (2004) Int J Dev Neurosci 22:205-213.
Mackay-Sim and Kittel (1991) J Neurosci 11:979-984.
Malgrange et al. (2002) Mech Dev 112:79-88.
Morest and Cotanche (2004) J Neurosci Res 78:455-460.
Murrell et al. (2005) Dev Dynamics 233:496-515.
Othman et al. (2003) Biotech Histochem 78:57-70.
Pau et al. (2005) Laryngoscope 115:116-124.
Shepherd et al. (2005) J Comp Neurol 486:145-158.
Stone and Rubel (2000) Proc Natl Acad Sci USA 97:11714-11721.
Raphael (2002) Br Med Bull 63:25-38.
Roisen et al. (2001) Brain Res 890:11-22.
White et al. (2006) Nature 441:984-987.

1. A method of generating cochlear hair cell-like cells in the inner ear of a subject, comprising administering adult olfactory precursor cells, or progeny thereof, to the inner ear of the subject.

2. The method of claim 1, wherein the subject is human.

3. The method of claim 2, wherein the human is suffering sensorineural hearing loss.

4. The method of claim 1, wherein the adult olfactory precursor cells are autologous.

5. The method of claim 1, wherein the adult olfactory precursor cells, or progeny thereof, are administered to the cochlea.

6. The method of claim 5, wherein the adult olfactory precursor cells, or progeny thereof, are administered to the cochlea by contact with the scala tympani via round window penetration.

7. The method of claim 5, wherein the adult olfactory precursor cells, or progeny thereof, are administered to the cochlea through the external auditory canal and the middle ear.

8. The method of claim 1, wherein the adult olfactory precursor cells are isolated from the nasal cavity.

9. The method of claim 1, wherein the progeny of adult olfactory precursor cells form neurospheres.

10. A method of generating cochlear hair cell-like cells in vitro, comprising culturing adult olfactory precursor cells, or progeny thereof, with cochlear cells, or with supernatant derived therefrom, such that the olfactory precursor cells, or progeny thereof, differentiate into cochlear hair cell-like cells.

11. The method of claim 10, wherein the adult olfactory precursor cells, or progeny thereof, and the cochlear cells, or supernatant derived therefrom, are co-cultured for 7 to 14 days.

12. The method of claim 10, wherein the adult olfactory precursor cells, or progeny thereof, and the cochlear cells are separate during the culturing phase.

13. The method of claim 10, wherein the cochlear hair cell-like cells are Myosin Vila-positive, calretinin-positive, phalloidin-positive, epsin-positive, and β III tubulin-negative.

14. Cochlear hair cell-like cells obtained by the method according to claim 10.

15. A method of treating or preventing an inner ear hair cell-related condition, comprising administering to the inner ear of the subject in need of such treatment the cochlear hair cell-like cell culture of claim 14.

16. The method of claim 15, wherein the inner ear hair cell-related condition is sensorineural hearing loss.
